(19) United States
(12) Patent Application Publication
(10) Pub. No.: US 2005/0042200 A1
(43) Pub. Date: Feb. 24, 2005

(54) Method and composition for inducing weight loss

(76) Inventors: James C. Keith, Andover, MA (US); Theo L. Peeters, Linden (BE); Inge Depoortere, Bonheiden (BE); Gert Van Assche, Leuven (BE)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/863,709

(22) Filed: Jun. 7, 2004

Related U.S. Application Data
(63) Continuation of application No. PCT/US02/39170, filed on Dec. 6, 2002.
(60) Provisional application No. 60/338,327, filed on Dec. 6, 2001.

(51) Int. Cl. A61K 38/20

(52) U.S. Cl. 424/85.2

(57) ABSTRACT

The use of interleukin-11 to prevent, to ameliorate, and to induce weight loss and/or to increase levels of leptin in a mammal, including an adult mammal, in need of such treatment are disclosed.

### FIELD OF THE INVENTION

The invention relates generally to methods and compositions for inducing weight loss in a subject.

### BACKGROUND OF THE INVENTION

Obesity is a common medical disorder that can adversely affect an afflicted individual's lifestyle. Moreover, obesity can greatly increase the risk of developing conditions such as high blood pressure and diabetes.

Some regulators of obesity have been identified through genetic analyses. For example, mice that are homozygous for the ob gene (ob/ob) are obese. The product of the ob gene has been identified and named leptin. Administration of leptin to ob/ob mice results in a reduction in body weight and food intake.

Leptin is primarily secreted by adipose tissue, and exerts its effects by interactions with specific receptors, e.g. in the hypothalamus. In humans, circulating leptin levels are increased in obesity and regulated by fasting, feeding, and body weight changes.

### SUMMARY OF THE INVENTION

The invention is based in part on the discovery that administration of interleukin-11 (“IL-11”) results in increased levels of leptin and weight loss in an adult subject. Accordingly, the invention provides compositions and methods for inducing weight loss by administering to subject agents that increase interleukin-11 levels in the subject.

In one aspect, the invention provides a method for inducing weight loss in a mammal by administering to the mammal interleukin-11 in an amount sufficient to increase leptin levels in the mammal.

The method optionally includes the step of identifying a mammal in which an increase in leptin is desired. In addition, the method optionally includes the step of detecting leptin levels (e.g., serum leptin levels). Leptin levels can be detected before and/or after administration of interleukin-11.

The effective amount of interleukin-11 can be, e.g., 1-to 1000 μg/kg body weight.

In some embodiments, the interleukin-11 is administered at least twice over a period of 24 hours or more to the subject.

In other embodiments, the interleukin-11 is administered daily to the subject.

The mammal can be, e.g., a human, a non-human primate, a rodent (such as a mouse or rat), a dog, cat, horse, cow, pig, or goat.

Administration can be topical or systemic. Modes of administration can include, subcutaneous, intravenous or oral administration.

In some embodiments, the mammal has or is at risk for diabetes.

In some embodiments, the mammal is substantially free of dividing adipocytes.

In some embodiments, the mammal is substantially free of differentiating adipocytes.

In another aspect, the invention provides a method of increasing leptin levels in a mammal, comprising administering to the mammal an effective amount of interleukin-11.

The method optionally includes the step of identifying a mammal in which an increase in leptin is desired. In addition, the method optionally includes the step of detecting leptin levels (e.g., serum leptin levels). Leptin levels can be detected before and/or after administration of interleukin-11.

The effective amount of interleukin-11 can be, e.g., 1-to 1000 μg/kg body weight.

In some embodiments, the interleukin-11 is administered at least twice over a period of 24 hours or more to the subject.

In other embodiments, the interleukin-11 is administered daily to the subject.

The mammal can be, e.g., a human, a non-human primate, a rodent (such as a mouse or rat), a dog, cat, horse, cow, pig, or goat.

Administration can be topical or systemic. Modes of administration can include, subcutaneous, intravenous or oral administration.

In some embodiments, the mammal has or is at risk for diabetes.

In some embodiments, the mammal is substantially free of dividing adipocytes.

In some embodiments, the mammal is substantially free of differentiating adipocytes.

In a further aspect, the invention provides a method of increasing satiety in a mammal, the method comprising administering to a mammal in which an increase in satiety is desired an effective amount of interleukin-11.

The method optionally includes the step of identifying a mammal in which an increase in leptin is desired. In addition, the method optionally includes the step of detecting leptin levels (e.g., serum leptin levels). Leptin levels can be detected before and/or after administration of interleukin-11.

The effective amount of interleukin-11 can be, e.g., 1-to 1000 μg/kg body weight.

In some embodiments, the interleukin-11 is administered at least twice over a period of 24 hours or more to the subject.

In other embodiments, the interleukin-11 is administered daily to the subject.

The mammal can be, e.g., a human, a non-human primate, a rodent (such as a mouse or rat), a dog, cat, horse, cow, pig, or goat.

Administration can be topical or systemic. Modes of administration can include, subcutaneous, intravenous or oral administration.

In some embodiments, the mammal has or is at risk for diabetes.

In some embodiments, the mammal is substantially free of dividing adipocytes.

In some embodiments, the mammal is substantially free of differentiating adipocytes.

In a still further aspect, the invention provides a method of identifying an agent that increases leptin levels in a cell population. The method includes identifying an IL-11 receptor agonist, contacting a leptin-expressing cell population with the IL-11 receptor agonist, and determining whether the IL-11 receptor agonist alters the amount of leptin in the cell population. An increase in the amount of leptin in the contacted cell population indicates that the IL-11 receptor agonist is an agent that increases leptin levels in a cell population.

In some embodiments, the IL-11 receptor agonist includes an IL-11 receptor-binding region of an IL-11 polypeptide or an IL-11 receptor-binding region of an anti-IL-11 antibody. The cell population can include, e.g., adipocyte cells.

In another aspect, the invention provides a method of identifying an agent that increases leptin levels. The method includes administering to a subject an IL-11 receptor agonist and determining whether the IL-11 receptor agonist modulates leptin levels in the subject. The subject can be, e.g., a rodent or a rabbit. In one embodiment, leptin levels are determined by measuring plasma leptin levels in the subject.

In one aspect, the invention provides a method for inducing weight loss in a mammal by administering to the mammal a therapeutically effective amount of interleukin-11.

The therapeutically effective amount of interleukin-11 can be, e.g., 1-to 1000 μg/kg body weight. In some embodiments, the interleukin-11 is administered at least twice over a period of 24 hours or more to the subject until weight loss is observed. For example, the interleukin-11 can be administered daily to the subject until weight loss is observed. In some embodiments, IL-11 is administered to the mammal until the mammal loses 5% more weight than the mammal would lose without being administered IL-11. In other embodiments, IL-11 is administered to the mammal until the mammal loses 10, 15, 20, or 25% more weight than the mammal would lose without being administered IL-11.

The mammal can be, e.g., a human, a non-human primate, or a dog, cat, horse, or rabbit. In some embodiments, the mammal suffer from, or be at risk for, conditions associated with undesired weight gain, e.g., diabetes (including type I or type II diabetes).

In general, any route of administration can be used so that the IL-11 exerts its desired effects in the host. In preferred embodiments, the IL-11 is administered systemically to the mammal, e.g., subcutaneously, intravenously, or intramuscularly to the mammal.

Also provided by the invention is a method of increasing leptin levels in a mammal by identifying a mammal in which an increase of leptin levels (e.g., serum leptin levels) is desired, and administering to the mammal a therapeutically effective amount of interleukin-11. The therapeutically effective amount of interleukin-11 can be, e.g., 1-to 1000 μg/kg body weight. In some embodiments, the interleukin-11 is administered at least twice over a period of 24 hours or more to the subject until weight loss is observed. For example, the interleukin-11 can be administered daily to the subject until weight loss is observed. In some embodiments, IL-11 is administered to the mammal until leptin levels increase 10% relative to the same or similarly matched mammal not treated with IL-11. In other embodiments, IL-11 is administered to the mammal until the mammal leptin levels increase 25, 50, 100, or even 200%.

In another aspect, the invention provides a method for treating or preventing obesity in a subject by administering to the mammal a therapeutically effective amount of IL-11.

Also provided by the invention is a pharmaceutical composition that includes a pharmaceutically acceptable carrier and an amount of IL-11 sufficient to result in weight loss when introduced into a mammal.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patent, and other references mentioned herein are incorporated by reference in their entirety. In the case of conflict, the present Specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The invention is based in part on the unexpected observation that IL-11 can induce weight loss in an adult subject. Interleukin-11 (IL-11), which is also known as recombinant adipogenesis inhibitory factor (AGIF), has been previously reported to inhibit the differentiation of pre-adipocytes into adipocytes. However, because the number of adipocytes in an individual is thought to be completed by late adolescence, it was previously thought that the usefulness of IL-11 in inducing weight loss occurs by inhibiting formation of adipocytes and would thus be restricted to younger subjects, i.e., those in which differentiation of preadipocytes into adipocytes was still occurring. In contrast, IL-11 was not thought to be useful for inducing weight loss in a post-adolescent subject, in whom weight losses are typically attributed to changes in the amount of fat stored in pre-existing adipocytes. Accordingly, in one aspect the invention provides compositions and methods for inducing weight loss in a mammalian subject that is substantially free of dividing adipocytes. The subject can be, e.g., an adult mammal (such as a post-adolescent human).

While not wishing to be bound by theory, it is believed that administration of IL-11 leads to weight loss in a subject by increasing leptin levels. Leptin is thought to induce satiety, which can lead over time to weight loss in the subject.

IL-11 used in the methods and compositions disclosed herein can be obtained using methods known in the art. IL-11 is described in detail in International Application PCT/US90/06803, published May 30, 1991; as well as in U.S. Pat. No. 5,215,895, issued Jun. 1, 1993. A cloned human IL-11 was previously deposited with the ATCC, 10801 University Boulevard, Manassa, Va. 20110-2209, on Mar. 30, 1990 under ATCC No. 68284. Moreover, as described in U.S. Pat. No. 5,270,181; issued Dec. 14, 1993; and U.S. Pat. No. 5,292,646; issued Mar. 8, 1994; IL-11 may also be produced recombinantly as a fusion protein with another protein. IL-11 can be produced in a variety of host cells by resort to now conventional genetic engineering techniques. In addition, IL-11 can be obtained from various cell lines, for example, the human lung fibroblast cell line, MRC-5 (ATCC Accession No. CCL 171) and Paul et al., the human trophoblastic cell line, TPA30-1 (ATCC Accession No. CRL 1583). Described in Proc Natl Acad Sci USA 87:7512 (1990) is a cDNA encoding human IL-11 as well as the deduced amino acid sequence (amino acids 1 to 199). U.S. Pat. No. 5,292,646, supra, describes a des-Pro form of IL-11 in which the N-terminal proline of the mature form of IL-11 (amino acids 22-199) has been removed (amino acids 23-199). As is appreciated by one skilled in the art, any form of IL-11, which retains IL-11 activity, is useful according to the present invention.

In addition to recombinant techniques, IL-11 may also be produced by known conventional chemical synthesis. Methods for constructing the polypeptides useful in the present invention by synthetic means are known to those of skill in the art. The synthetically constructed cytokine polypeptide sequences, by virtue of sharing primary, secondary, or tertiary structural and conformational characteristics with the natural cytokine polypeptides are anticipated to possess biological activities in common therewith. Such synthetically constructed cytokine polypeptide sequences or fragments thereof, which duplicate or partially duplicate the functionality thereof may also be used in the method of this invention. Thus, they may be employed as biologically active or immunological substitutes for the natural, purified cytokines useful in the present invention.

Modifications in the protein, peptide or DNA sequences of these cytokines or active fragments thereof may also produce proteins for use in the methods and compositions disclosed herein. Modifications of the IL-11 sequence may include the replacement, insertion or deletion of one or more selected amino acid residues in the coding sequences. Mutagenic techniques for such replacement, insertion or deletion are well known to one skilled in the art. (See, e.g., U.S. Pat. No. 4,518,584.)

Other specific mutations of the sequences of the cytokine polypeptides that may be useful therapeutically as described herein may involve, e.g., the insertion of one or more glycosylation sites. An asparagine-linked glycosylation recognition site can be inserted into the sequence by the deletion, substitution or addition of amino acids into the peptide sequence or nucleotides into the DNA sequence. Such changes may be made at any site of the molecule that is modified by addition of O-linked carbohydrate. Expression of such altered nucleotide or peptide sequences produces variants that may be glycosylated at those sites.

Additional analogs and derivatives of the sequence of the selected cytokine which would be expected to retain or prolong its activity in whole or in part, and which are expected to be useful in the present method, may also be easily made by one of skill in the art. One such modification may be the attachment of polyethylene glycol (PEG) onto existing lysine residues in the cytokine sequence or the insertion of one or more lysine residues or other amino acid residues that can react with PEG or PEG derivatives into the sequence by conventional techniques to enable the attachment of PEG moieties.

Additional analogs may also be characterized by allelic variations in the DNA sequences encoding them, or induced variations in the DNA sequences encoding them. It is anticipated that all analogs disclosed in the above-referenced publications, including those characterized by DNA sequences capable of hybridizing to the disclosed cytokine sequences under stringent hybridization conditions or non-stringent conditions (Sambrook et al., Molecular Cloning. A Laboratory Manual, 2d edit. Cold Spring Harbor Laboratory, New York (1989)) will be similarly useful in this invention.

Also considered useful in these methods are fusion molecules, prepared by fusing the sequence or a biologically active fragment of the sequence of one cytokine to another cytokine or proteinaceous therapeutic agent, e.g., IL-11 fused to IL-6 (see, e.g., methods for fusion described in PCT/US91/06186 (WO92/04455), published Mar. 19, 1992). Alternatively, combinations of the cytokines may be administered together according to the method.

Thus, where in the description of the methods of this invention IL-11 is mentioned by name, it is understood by those of skill in the art that IL-11 encompasses the protein produced by the sequences presently disclosed in the art, as well as proteins characterized by the modifications described above yet which retain substantially similar activity.

Pharmaceutical compositions containing IL-11 which are useful in practicing the methods of the present invention may also contain pharmaceutically acceptable carriers, diluents, fillers, salts, buffers, stabilizers and/or other materials well-known in the art. The term “pharmaceutically acceptable” means a material that does not interfere with the effectiveness of the biological activity of the active ingredient(s) and that is not toxic to the host to which it is administered. The characteristics of the carrier or other material will depend on the route of administration.

In some embodiments, the pharmaceutical compositions contain about 0.1 micrograms to about 1 milligram per milliliter of the active ingredient.

Administration can be carried out in a variety of conventional ways. Intraperitoneal injection is the preferred method of administration. Intravenous, cutaneous or sub-cutaneous injection may also be employed. For injection, IL-11 will preferably be administered in the form of pyrogen-free, parenterally acceptable aqueous solutions. The preparation of such parenterally acceptable protein solutions, having due regard to pH, isotonicity, stability and the like, is within the skill of the art. IL-11 can additionally be administered orally. Oral preparations of IL-11 are disclosed in, e.g., WO02/22156 and Greenwood-Van Meerveld et al., J Pharmacol Exp Ther. 299:58-66, 2001.

The amount of IL-11 used for treatment will depend upon the severity of the condition, the route of administration, the reactivity or activity of the active ingredient, and ultimately will be decided by the treatment provider. In practicing the methods of treatment of this invention, a therapeutically effective amount of IL-11 is administered. The term “therapeutically effective amount” means the total amount of each active component of the method or composition that is sufficient to show a meaningful benefit in the subject (e.g., curing, ameliorating, inhibiting, delaying or preventing onset of, preventing recurrence or relapse of). One common technique to determine a therapeutically effective amount for a given subject is to administer escalating doses periodically until a meaningful benefit is observed. When applied to an individual active ingredient, administered alone, the term refers to that ingredient alone. When applied to a combination, the term refers to combined amounts of the active ingredients that result in the therapeutic effect, whether administered in combination, serially or simultaneously. A therapeutically effective dose of IL-11 in this invention is contemplated to be in the range of about 1 to about 1000 μg/kg body weight, and more preferably between about 1 and about 100 μg/kg body weight. The number of administrations may vary, depending on the individual patient and the severity of the gastrointestinal disorder.

Satiety can be measured using methods known in the art, including, e.g., by assessing the desire of a subject to consume food. Methods and apparatuses for assessing satiety are also discussed in U.S. Pat. No. 5,817,006.

Methods of Identifying an Agent that Increases Leptin Levels

Agents that increase leptin levels in a cell population are by identifying an IL-11 receptor agonist and contacting a leptin-expressing cell population with the IL-11 receptor agonist. The method additionally includes determining whether the IL-11 receptor agonist alters the amount of leptin in the population. An increase in the amount of leptin in the contacted cell population indicates that the IL-11 receptor agonist is an agent that increases leptin levels in a cell population.

An IL-11 receptor agonist as used herein is any agent that increases one or more activities of an IL-11 receptor polypeptide. IL-11 initiates signaling via binding to a unique IL-11-receptorα-(IL-11Rα) chain. Nandurkar, H. H., et al. (1996) Oncogene 12:585-593; Miyatake, T., et al. (1998) J. Immunol. 160:4114-4123. Nucleotide and encoded amino acid sequences of the human IL-11 receptor are disclosed in U.S. Pat. Nos. 6,350,855, and 6,274,547.

The activity of an IL-11 receptor polypeptide can include activities of polypeptides through which the IL-11 receptor polypeptide transduces a signal. The IL-11/IL-11 Rα complex is thought to bind to and induce clustering gp130, leading to the activation, via transphosphorylation, of associated JAKs. Yin, T., K., et al. (1994) Exp. Hematol. 22:467-472; Wang, X. Y., et al. (1995) J. Biol. Chem. 270:27999-28002. IL-11 belongs to the interleukin-6 (IL-6) family of cytokines, all of which use gp130 as a critical component for signal transduction. Taga, T. and T. Kishimoto (1997) Annu. Rev. Immunol. 15:797-819; Zhang, X. G., et al. (1994) J. Exp. Med. 179:1337-1342; and Yang, Y. C. and T. Yin (1995) Ann. N.Y. Acad. Sci. 762:31-40. Activated JAKs phosphorylate tyrosine residues within the cytoplasmic region of gp 130, which then serve as docking sites for signal transducer and activators of transcription proteins, STAT3 and STAT1. Lutticken, C., et al. (1994) Science 263:89-92; Hemmann, U., et al. (1996) J. Biol. Chem. 271:12999-13007. The activated JAKs subsequently phosphorylate tyrosine residues within the bound STAT proteins, causing the STATs to dissociate from gp 130, dimerize, and enter the nucleus to act as transcriptional activators of target genes. Zhong, Z., et al. (1994) Science 264:95-98; Ihle, J. N. (1996) Cell 84:331-334; and Akira, S. (1997) Int. J. Biochem. Cell Biol. 29:1401-1418. STAT dimers may be additionally phosphorylated on serine or threonine residues by mitogen activated protein kinases (MAPKs) that are also activated in response to cytokine binding to the receptor. Zhang, X., et al. (1995) Science 267:1990-1994; Boulton, T. G., et al. (1995) Proc. Natl. Acad. Sci. U.S.A. 92:6915-6919; Adunyah, S. E., et al. (1995) Ann. N.Y. Acad. Sci. 766:296-299; and Yin, T. and Y. C. Yang (1994) J. Biol. Chem. 269:3731-3738. This additional phosphorylation may potentiate STAT function as an activator of transcription.

The activity of an IL-11 receptor agonist may additionally be assessed using one or more activities of an IL-11 polypeptide. IL-11 is a stromal cell-derived pleiotropic cytokine that interacts with a variety of hematopoietic and non-hematopoietic cell types. Recombinant human IL-11 stimulates megakaryocytopoiesis in vitro and in vivo. Weich, N. S., et al. (1997) Blood 90:3893-3902; and Orazi, A., et al. (1996) Exp. Hematol. 24:1289-1297. IL-11 also stimulates erythropoiesis and regulates macrophage proliferation and differentiation. de Haan, G., et al. (1995) Br. J. Haematol. 90:783-790. Due to its thrombopoietic activities in vivo, IL-11 is used to treat chemotherapy-induced thrombocytopenia. Kaye, J. A. (1996) Curr. Opin. Hematol. 3:209-215.

In addition to its hematopoietic effects, IL-11 also protects against various forms of mucosal epithelial cell injury. For example, IL-11 has been shown to protect small intestinal cells from combined radiation, chemotherapy, and ischemia (Du, X., et al. (1997) Am. J. Physiol. 272:G545-G552; Orazi, A., et al. (1996) Lab. Invest. 75:33-42; and Keith, J. C., Jr., et al. (1994) Stem. Cells. (Dayt). 1(12):79-89); reduce experimental colitis induced by trinitrobenzene sulfonic acid in rat (Qiu, B. S., et al. (1996) Dig. Dis. Sci. 41:1625-1630); and ameliorate inflammatory bowel disease (Orazi, A., et al. (1996) Lab. Invest. 75:33-42). The foregoing studies show that treatment with IL-11 decreases mucosal damage, accelerates healing and improves host survival. IL-11 also reduces immune-mediated small bowel injury in acute GVHD following murine allogeneic bone marrow transplantation. Hill, G. R., et al. (1998) J. Clin. Invest. 102:115-123.

Changes in leptin levels can be assessed by measuring either levels of leptin RNA or leptin polypeptides. The nucleotide and encoded polypeptide sequences of leptons from several mammalian species are known, including human and mouse leptin (Zhang et al., Nature 372:425-32, 1994), porcine leptin (U.S. Pat. No. 6,277,592), and bovine leptin (U.S. Pat. No. 6,297,027). Leptin antibodies are commercially available (e.g., Research & Diagnostic Antibodies, Benicia, Calif., Abcam, Ltd., Cambridge, UK). Accordingly, leptin RNA can be determined using any art-recognized method for detecting nucleic acid sequences. One suitable method is RT-PCR. Leptin polypeptides can be measured using methods known in the art, e.g., radioimmune assays using anti-leptin polypeptides.

Sources of IL-11 receptor agonists can include, e.g., an IL-11 receptor binding portion of an IL-11 polypeptide, or a polypeptide that includes an IL-11 receptor-binding region of an anti-IL-11 receptor antibody.

A preferred cell type is a cell that expresses an IL-11 receptor and is capable of expressing leptin. In some embodiments, the cell population includes adipocyte cells.

Also within the invention are methods of identifying an agent that increases leptin levels by administering to a subject (such as a rodent or rabbit) an IL-11 receptor agonist and measuring the leptin levels in the subject. A preferred method of measuring leptin levels is to measure plasma leptin levels.

The invention will be further illustrated in the following non-limiting example.

### EXAMPLE 1

Effect of IL-11 on Leptin Levels and Weight Loss

The effect of IL-11 on levels of the satiety inducing peptide, leptin, in non-inflamed and inflamed rabbits was examined. The effect of leptin on motilin expression was also examined.

Rabbits received 4, 40, 72, or 720 μg/kg rhIL-11 or saline administered subcutaneously. One hour later a continuous subcutaneous administration of rhIL-11 was started with or without the induction of colitis (135 mg/kg TNBS) for 5 days. Plasma leptin and motilin levels were measured by RIA before and 5 days after the induction of colitis. The effect of leptin (10 ng/ml) on motilin expression was determined by competitive PCR in T84 cells.

Reference values for plasma leptin were first established in fed and in fasted (90 hours) rabbits. Fasting decreased leptin from 2497±408 pg/ml to 1615±123 pg/ml, and was accompanied by a body weight loss of 135±37 gram (−4.7±1.5%). Five days after the induction of colitis, rabbits lost 317±13 gram of body weight, which corresponds to 11.8±1.0% of their weight. Plasma leptin and motilin levels were unaffected.

During treatment with IL-11 weight loss was enhanced to 15.9±1.4% (4 μg/kg per day IL-11) at day 5. Higher doses of IL-11 did not worsen this effect (720 μg/kg per day IL-11 ; −17.1±1.2%. However, IL-11 treatment dose-dependently increased plasma leptin levels with 677±216 pg/ml per day IL-11 (36±9%) (4 μg/kg per day IL-11) and 2084±797 pg/ml (256±97%) (720 μg/kg per day). In non-inflamed animals, a prompt decrease in weight (−12.1±1.2%) was observed with the highest dose of IL-11. At this dose plasma leptin and motilin levels increased to 997±156 pg/ml (69±18%) and 590±30 pg/ml (113±14%).

In order to investigate whether the effect of IL-11 on motilin release is mediated by the release of leptin, T84 cells were stimulated with leptin, and the effect on motilin expression was investigated by PCR. At 1.5 hours and 48 hours after stimulation with 10 ng/ml leptin, motilin was increased 2.27±0.47 and 2.66±0.75 fold, respectively. In both inflamed and non-inflamed rabbits, IL-11 treatment dose-dependently increased plasma leptin levels. One explanation for these results is that IL-11 interacts with the leptin receptor to stimulate leptin release. The leptin-mediated increase in motilin expression may be part of a feedback mechanism.

While the present invention has been described in terms of specific methods and compositions, it is understood that variations and modifications will occur to those skilled in the art upon consideration of the present invention. Numerous modifications and variations in the invention as described in the above illustrative examples are expected to occur to those skilled in the art and, consequently, only such limitations as appear in the appended claims should be placed thereon. Accordingly, it is intended in the appended claims to cover all such equivalent variations that come within the scope of the invention as claimed.

1. A method for inducing weight loss in a mammal, comprising administering to the mammal interleukin-11 in an amount sufficient to increase leptin levels in the mammal.

2. A method of increasing leptin levels in a mammal, comprising administering to the mammal an effective amount of interleukin-11.

3. A method of increasing satiety in a mammal, the method comprising administering to a mammal in which an increase in satiety is desired an effective amount of interleukin-11.

4. The method of claim 1, 2, or 3, further comprising the step of identifying a mammal in which an increase in leptin is desired.

5. The method of claim 1, 2, or 3, further comprising the step of detecting leptin levels before and after administration of interleukin-11.

6. The method of claim 1, 2, or 3, wherein the effective amount of interleukin-11 comprises 1-to 1000 μg/kg body weight.

7. The method of claim 1, 2, or 3, wherein the interleukin-11 is administered at least twice over a period of 24 hours or more to the subject.

8. The method of claim 1, 2, or 3, wherein the interleukin-11 is administered daily to the subject.

9. The method of claim 1, 2, or 3, wherein the mammal is a human.

10. The method of claim 1, 2, or 3, wherein the IL-11 is administered systemically to the mammal.

11. The method of claim 1, 2, or 3, wherein the IL-11 is administered subcutaneously or intravenously.

12. The method of claim 1, 2, or 3, wherein the IL-11 is administered orally.

13. The method of claim 1, 2, or 3, wherein the mammal has or is at risk for diabetes.

14. The method of claim 1, 2, or 3, wherein the mammal is substantially free of dividing adipocytes.

15. The method of claim 1, 2, or 3, wherein the mammal is substantially free of differentiating adipocytes.

16. The method of claim 2, wherein the leptin levels are serum leptin levels.

17. A method of identifying an agent that increases leptin levels in a cell population, the method comprising
identifying an IL-11 receptor agonist;
contacting a leptin-expressing cell population with the IL-11 receptor agonist; and
determining whether the IL-1 receptor agonist alters the amount of leptin in the cell population, wherein an increase in the amount of leptin in the contacted cell population indicates that the IL-11 receptor agonist is an agent that increases leptin levels in a cell population.

18. The method of claim 17, wherein the IL-11 receptor agonist comprises an IL-11 receptor binding region of an IL-11 polypeptide.

19. The method of claim 17, wherein the IL-11 receptor agonist comprises an IL-11 receptor-binding region of an anti-IL-11 antibody

20. The method of claim 17, wherein the cell population comprises adipocyte cells.

21. A method of identifying an agent that increases leptin levels, the method comprising
administering to a subject an IL-11 receptor agonist; and
determining whether the IL-11 receptor agonist modulates leptin levels in the subject.

22. The method of claim 21, wherein the subject is a rodent or a rabbit.

23. The method of claim 21, wherein leptin levels are determined by measuring plasma leptin levels in the subject.
